(19) United States
(12) Patent Application Publication
(10) Pub. No.: US 2010/0113871 A1
(43) Pub. Date: May 6, 2010

(54) ANTIMICROBIAL COATING

(76) Inventors: Aylvin Jorge Angelo Athanasius Dias, Maastricht (NL); Edith Elisabeth M. Van Den Bosch, Riemst (BE); Astrid Franken, Linnich (DE)

Correspondence Address: NIXON & VANDERHYE, PC, 901 NORTH GLEBE ROAD, 11TH FLOOR, ARLINGTON, VA, 22203, US

(21) Appl. No.: 12/440,543

(22) PCT Filed: Sep. 13, 2007
(86) PCT No.: PCT/EP07/07995

§ 371 Date: Dec. 8, 2009

(30) Foreign Application Priority Data
Sep. 13, 2006 (EP) 06019149.1

(51) Int. Cl. A61L 29/16; A61L 31/16; A41D 19/015; A61L 15/00; A61L 17/00

(52) U.S. Cl. 600/101; 604/265; 128/844; 128/207.14; 2/161.7; 602/48; 606/301; 606/151; 606/231; 424/618; 427/2.24; 427/2.3

(57) ABSTRACT

The invention relates to a formulation for preparing an antimicrobial lubricious hydrophilic coating, which formulation comprises a hydrophilic polymer; an initiator; particles comprising metallic silver (i.e. Ag°); and a carrier liquid. The invention further relates to an article comprising a hydrophilic coating on a surface wherein the coating comprises a cured hydrophilic polymer and particles comprising metallic silver.

The invention relates to a formulation for preparing a hydrophilic coating comprising an antimicrobial agent, to a method for coating an article and to a coated article, in particular a medical device such as a catheter.

Infections that arise as a result of temporary or permanent implantations are some of the most serious and frequent sources of complications that arise from the use of invasive medical devices. During the implantation or insertion procedure of medical articles like catheters and vascular devices the mucosal or endothelial or indeed any biological counter surface is often damaged, resulting in microbial infections. Thus in the drive to minimise microbial infections it is important to combine lubricity, maintenance of lubricity (dry-out time), robustness (wear resistance) as well as the desired antimicrobial properties. Loss of lubricity in hydrogel coatings can occur due to the premature drying of the hydrogel which is accompanied by loss of lubricious properties and resultant damage to the biological counter surface.

Articles, in particular medical devices provided with an antimicrobial agent have been disclosed in several publications. Besides organic antimicrobial agents, ionic silver has also been reported as an antimicrobial agent. For instance, WO 02/07002 describes a method for providing a surface with a lubricious anti-microbial coating comprising silver ions or another anti-microbial agent. The coating described in WO 02/07002 is prepared by first providing a surface with a polymeric layer. Thereafter the polymeric layer is treated to allow binding of the silver. It is not disclosed to provide an article with a coating comprising metallic silver, let alone metallic silver particles, nor to form the polymeric layer in the presence of silver.

US 2003/0044451 describes a flexible coating comprising silicone and urethane, which is thermally cured. It is mentioned that the coating may comprise an antimicrobial agent, e.g. a silver salt. US 2003/0044451 does not reveal how to provide a coating with metallic silver, nor is a lubricious coating comprising metallic silver disclosed.

US 2001/0051669 relates to a medical article lubricant composition. Amongst others the composition comprises an isocyanate-terminated prepolymer, a polymer and a pharmacological additive. The additive may be an anti-microbial agent, such as silver. The coating procedure involves thermally curing the prepolymer.

A medical device coated with metallic silver is disclosed in US 2002/0094322. The silver is applied as a first layer on a substrate. This layer is overlaid with a second layer: a hydrogel, which contains an organic antimicrobial agent, such as chlorhexidine. The hydrogel serves to reduce friction. This method is rather complex as it requires separate coating steps for providing silver and for providing lubricity. Furthermore, the use of photo-initiators is not described.

In US 2003/0198821 it is reported that it is difficult to control the amount of silver deposited or retained when directly deposited on medical devices. It is also stated that it is difficult to control the release of silver from the surface of the article, making accurate and sustained dosing difficult. US 2003/0198821 proposes to coat silicone catheters with a primer layer comprising a silver salt colloid. Furthermore, a silane polymer coating is applied. This coating step involves thermal curing.

US 2005/0004525 relates to connecting an accessory between a urinary catheter and a leg bag. The accessory comprises a sleeve in which a filter is present that comprises an antimicrobial composition. Furthermore, the inside of the sleeve may be coated with an antimicrobial coating. The antimicrobial composition may comprise nano size particles of silver. This publication does not disclose a coating comprising a hydrophilic polymer that is cured by photo-initiation.

A lubricant may be present to make the surface lubricious, to the extent that cell adhesion is discouraged, i.e. the lubricant contributes to reduce fouling. This publication does not address an article comprising a coating that is lubricious in a mechanical sense, i.e. that the wear resistance is improved such that an article—in particular a catheter—can be inserted in a patient, for instance in a blood vessel or urinary tract, without causing serious damage to the tissue it is in contact with when it is being inserted.

Commercially available silver coated Foley catheters are sold by Bardex and by Tyco Kendall. As illustrated by the Examples below, it has been found that these catheters have an undesirably low lubricity after wetting, a short dry-out time and/or a low wear resistance. Further, it has been found that the lubricity varies a lot from catheter to catheter. It is thought that the presence of silver particles makes the coating rougher, and thereby less lubricious. Moreover, a release test, determining the release of ionic silver into an aqueous environment, using graphite furnace atomic absorption spectrometry (GF-AAS), revealed that the dual coated Bardex catheter did not release detectable amounts of silver. The dual coated Tyco Kendall catheter showed a relatively low silver release.

It is an object of the present invention to provide a novel formulation for providing an article with an antimicrobial coating, a novel method of coating an article with such a formulation, respectively a novel article provided with an antimicrobial coating.

It is a further object to provide such formulation, method respectively article, wherein both lubricity and antimicrobial activity are provided, preferably by a single functional layer.

It is a further object to provide a method that enables coating of all surfaces of an article, i.e. the internal and external surfaces.

It is a further object to provide such formulation which can also be used to coat an article at a relatively low temperature, for instance room temperature. This would in particular be desirable for an article coated with a coating that comprises a heat-sensitive component and/or an article that has a relatively low thermal stability, in particular an article of which a property is detrimentally affected at a temperature typically used for thermally curing and/or heat-drying a coating. Examples of such articles are articles made from a material that is not sufficiently dimensionally or mechanically stable at an elevated temperature (such as an article that melts or becomes too plastic) or an article that is not sufficiently chemically stable at an elevated temperature, such as an article made from a material that degrades, is oxidised or wherein heat causes blooming of a component in the material on a surface of the article.

It is a further object to provide a formulation for coating an article, respectively a coated article, from which silver can be released for a long period of time and/or from which silver can be released in a controlled manner.

One or more objects which may be solved in accordance with the invention will be apparent from the remainder of the description and/or the claims.

It has now been found that one or more objects underlying the invention are met, by providing a specific formulation, in particular a formulation comprising a hydrophilic polymer which can be cured in a specific way.

Accordingly, the present invention relates to a formulation for preparing an antimicrobial hydrophilic coating, which formulation comprises a hydrophilic polymer; a photo-initiator; particles comprising metallic silver (i.e. Ag°); and a carrier liquid.

The invention further relates to a method for preparing a coated article, comprising applying a formulation according to the invention to at least one surface of the article; and allowing the polymer to cure by exposing the formulation to electromagnetic radiation thereby activating the photo-initiator.

The invention further relates to an article comprising a hydrophilic coating on a surface, in particular a coated article obtainable by a method according to the invention, wherein the coating comprises a cured hydrophilic polymer and particles comprising metallic silver)(Ag°).

The invention further relates to a formulation of the invention, for medical use. In particular, the formulation may be used in the manufacture of a composition—in particular a coating—to reduce the risk of infections, for example catheter associated infections, such as catheter associated urinary tract infections and catheter associated blood stream infections, or for the treatment of a disorder selected from the group consisting of complications of the urinary tract, complications of a cardiovascular vessel, kidney infections, blood infections (septicaemia), urethral injury, skin breakdown, bladder stones and hematuria.

The invention further relates to the use of a formulation according to the invention or a coating obtainable by curing a formulation according to the invention to reduce bacterial adhesion or to act as a bacteriocidal agent. The formulation or coating may be used in vitro or in vivo.

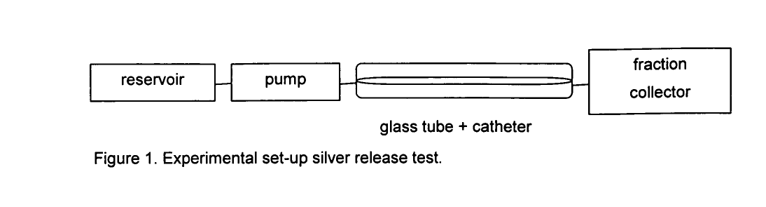

FIG. 1 is a schematic representation of a set-up used to determine the silver ion release from coated catheters.

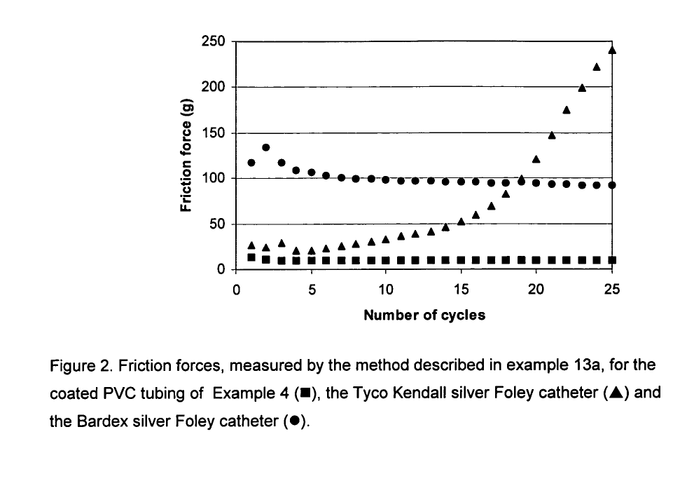

FIG. 2 is a comparison of the friction force of a coated catheter of the invention and two commercially available catheters.

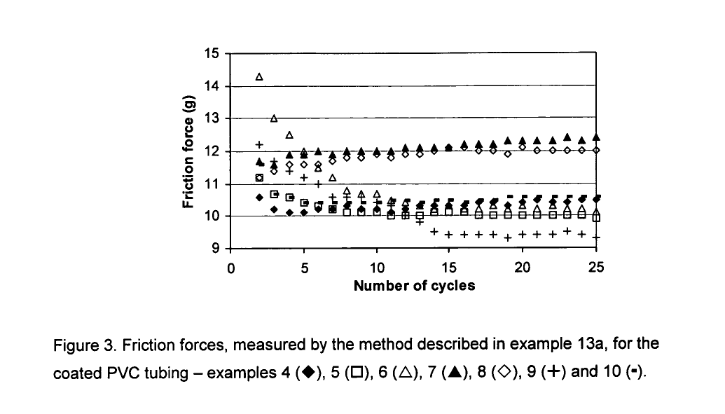

FIG. 3 shows the friction force for several coated catheters of the invention.

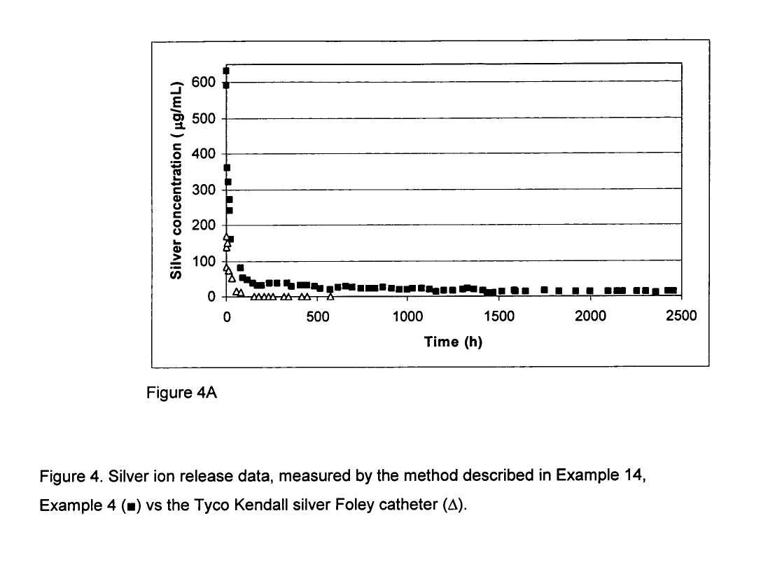

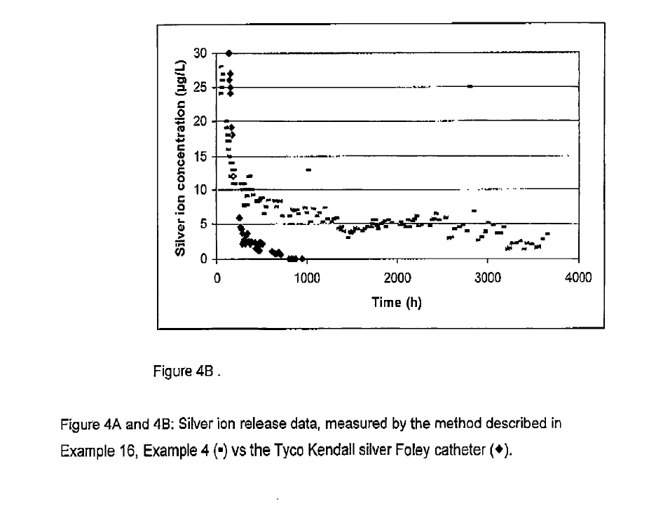

FIGS. 4A and 4B show silver release data as a function of time for a coated catheter of the invention and one commercially available catheter.

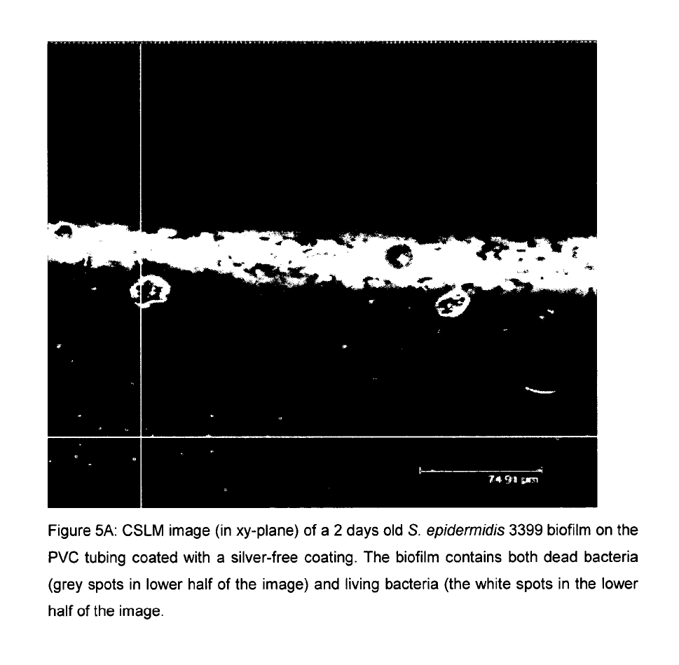

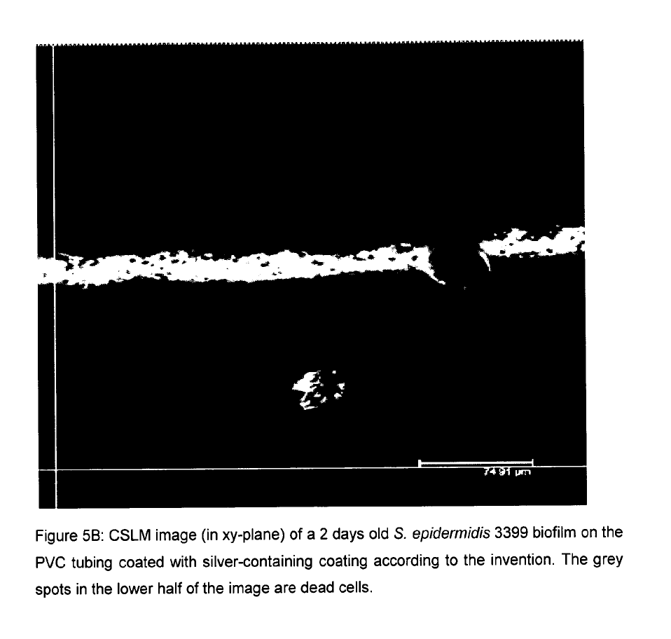

FIG. 5A shows a CSLM image (in xy-plane) of a 2 days old S. epidermidis 3399 biofilm on a PVC tubing coated with a silver-free coating

FIG. 5B shows a CSLM image (in xy-plane) of a 2 days old S. epidermidis 3399 biofilm on a PVC tubing coated with a silver-containing coating according to the invention.

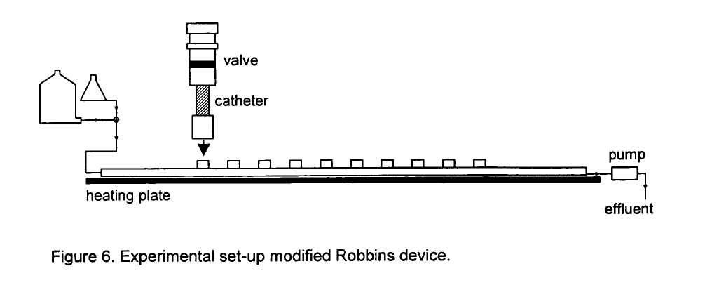

FIG. 6 schematically shows a modified Robbins Device.

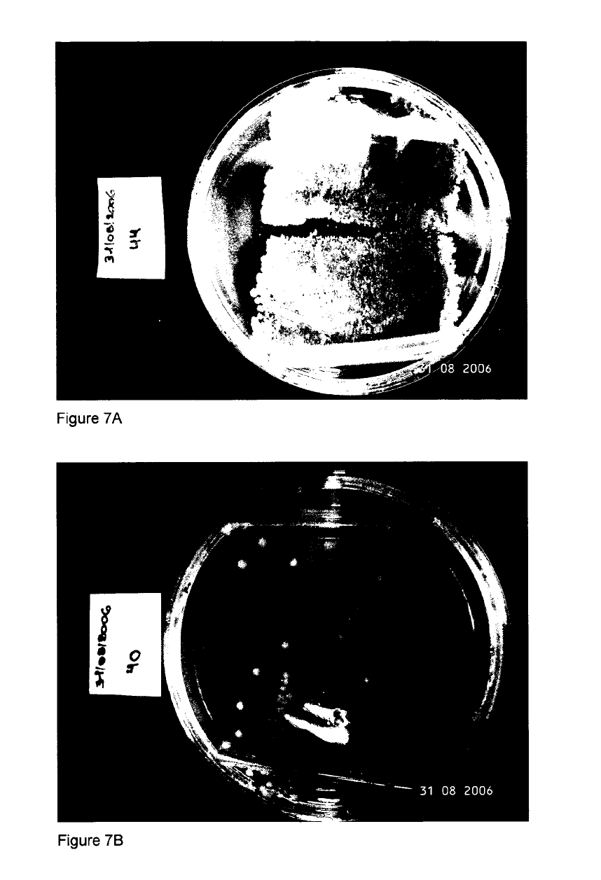

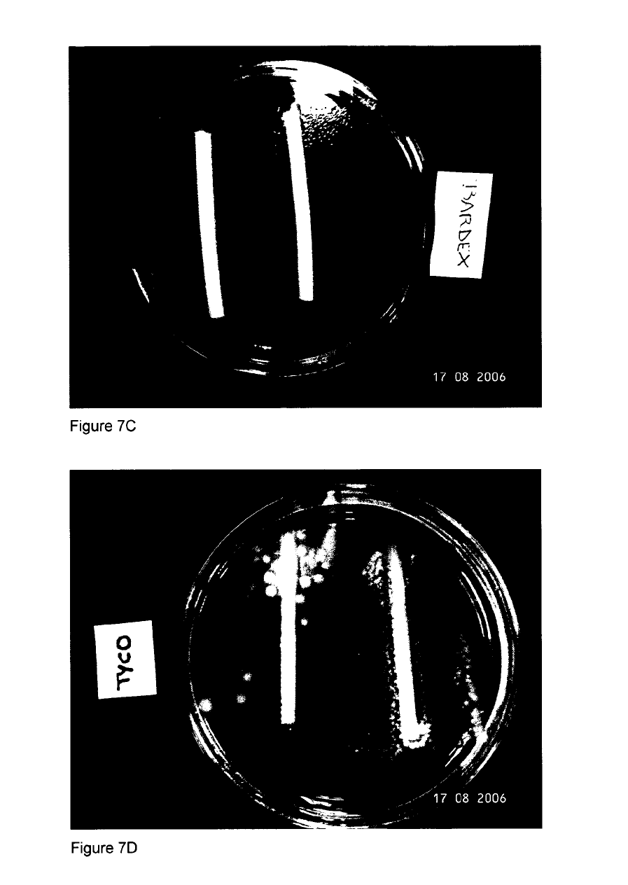

FIGS. 7A to D are photographs illustrating the antimicrobial activity of a coating in accordance with the invention compared to a silver-free coating and two commercially available coatings, comprising silver.

Typically, in an article of the invention the particles comprising the silver are dispersed in the polymer. It is surprising that it is possible to provide a formulation which is suitable to provide a coating wherein a single layer both provides (i) antimicrobial activity imparted by the presence of particles of metallic silver (ii) sufficient or even improved lubricity (or a high wear resistance) for insertion into a animal, including a human, without causing an unacceptable level of discomfort to the subject or damage to the tissue against which the article is moved during insertion; and wherein (iii) if needed the coating has a sufficiently long dry-out time to facilitate insertion/implantation into a subject. After all, the inclusion of particulate matter in a lubricious coating is generally considered to be detrimental to mechanical lubricity and/or wear resistance.

It is in particular surprising that it is possible to provide such coating by making use of photo-initiation to cure the polymer. It is unexpected that an advantageous antimicrobial and lubricious coating comprising metallic silver is thus obtained, as metallic silver is a known photo-active material.

The inventors have realised that providing a coating making use of a photo-initiator is advantageous in that it allows the coating of articles comprising a material that is not sufficiently thermally stable to allow thermal curing and/or drying at an elevated temperature.

The inventors further contemplate that also for coating an article which is thermally stable, thermal curing/drying may be disadvantageous. It is contemplated that as a result of the heating, one or more additives in the article—in particular one or more plasticizers may migrate to the surface of the article, possibly even into or through the coating, thereby affecting a property of the coating and/or leading to medical complications, in case the article is inside a patient's body or in contact therewith. For instance, blooming may occur as a result of migration of a plasticizer to the surface of the article. As a formulation may also be used to provide a coating without needing elevated temperature, such risk is avoided or at least reduced in a method of the invention.

It is further contemplated that the photo-curing provides an advantageous polymer network, in particular such network comprising grafts and/or cross-links, with good lubricity and/or wear resistance, also in the presence of the particles comprising silver.

Further, it has been found that a formulation of the invention is suitable to provide an article with an antimicrobial coating with a prolonged release of ionic silver, compared to a silver coated article according to the prior art, such as a commercially available catheter comprising silver.

It has further been found that it is possible to provide a coating from which ionic silver is released with a substantially zero-order release pattern (at least after a relatively short initial period needed to reach such release) for a considerable period of time (e.g. about 1000 hours or more). See e.g. FIG. 4, wherein is shown that a catheter of the invention shows substantially zero-order release in the period between 150 hrs and 2500 hrs after starting to release silver ions from the coating.

The term “polymer” is used herein for a molecule comprising two or more repeating units. In particular it may be composed of two or more monomers which may be the same or different. As used herein, the term includes oligomers and prepolymers. Usually polymers have a number average weight of about 500 g/mol or more, in particular of about 1000 g/mol or more, although the molar mass may be lower in case the polymer is composed of relatively small monomeric units and/or the number of units is relatively low. The term polymer includes oligomers. A polymer is considered an oligomer if it has properties which do vary significantly with the removal of one or a few of the units.

The term “to cure” includes any way of treating the formulation such that it forms a firm or solid coating. In particular, the term includes a treatment whereby the hydrophilic polymer further polymerises, is provided with grafts such that it forms a graft polymer and/or is cross-linked, such that it forms a cross-linked polymer.

In line with common practice, when referred to “a” moiety or “the” moiety (e.g. a compound for instance a (hydrophilic) polymer, a polyelectrolyte, an initiator) this is meant to refer to one or more species of said moiety.

Within the context of the invention a coating on the (outer) surface of a medical device, such as a catheter, is considered lubricious if (when wetted) it can be inserted into the intended body part without leading to injuries and/or causing unacceptable levels of pain to the subject. In particular, a coating is considered lubricious if it has a friction as measured on a Harland FTS Friction Tester of 20 g or less at a clamp-force of 300 g and a pull speed of 1 cm/s, preferably of 15 g or less. The protocol is as indicated in the Examples.

The term “wetted” is generally known in the art and—in a broad sense—means “containing water”. In particular the term is used herein to describe a coating that contains sufficient water to be lubricious. In terms of the water concentration, usually a wetted coating contains at least 10 wt. % of water, based on the dry weight of the coating, preferably at least 50 wt. %, based on the dry weight of the coating, more preferably at least 100 wt. % based on the dry weight of the coating. For instance, in a particular embodiment of the invention a water uptake of about 300-500 wt. % water is feasible.

Within the context of the invention, the dry-out time is the duration of the coating remaining lubricious after the device has been taken out of the wetting fluid wherein it has been stored/wetted. Dry-out time can be determined by measuring the friction in gram as a function of time the catheter had been exposed to air (22° C., 35% RH) on the Harland Friction tester. The dry-out time is the point in time wherein the friction reaches a value of 20 g or higher, or in a stricter test 15 g or higher.

As a hydrophilic polymer in principle any polymer may be used that is suitable to provide a lubricious hydrophilic coating. In particular, suitable is such a polymer that is polymerisable, graftable and/or cross-linkable in the presence of a photo initiator.

Generally such hydrophilic polymer may have a number average molar mass in the range of about 1 000-5 000 000 g/mol. Preferably the molar mass is at least, 20 000, more preferably at least 100 000. Advantageously, the molar mass is up to 2 000 000, in particular up to 1 300 000 g/mol. The molar mass is the value as determined by light scattering.

The polymer may for instance be a prepolymer, i.e. a polymer comprising one or more polymerisable groups, in particular one or more radically polymerisable groups such as one or more vinyl groups.

For providing a cross-linked network, a prepolymer having an average number of reactive groups per molecule of more than 1 is in particular suitable. Preferably, the average number of reactive groups is at least 1.2, more preferably at least 1.5, in particular at least 2.0. Preferably the average number of groups is up to 64, more preferably in the range of up to 15, in particular in the range of up to 8, more in particular up to 7.

However, also a polymer which is free of such polymerisable groups may be cured in the presence of a photo-initiator, in particular by the formation of grafts when the formulation is exposed to light.

In preferred embodiment, the formulation comprises at least one hydrophilic polymer selected from the group consisting of poly(lactams), in particular polyvinylpyrrolidones; polyurethanes; homo- and copolymers of acrylic and methacrylic acid; polyvinyl alcohols; polyvinylethers; maleic anhydride based copolymers; polyesters; vinylamines; polyethyleneimines; polyethylene oxides; poly(carboxylic acids); polyamides; polyanhydrides; polyphosphazenes; cellulosics, in particular methyl cellulose, carboxymethyl cellulose, hydroxymethylcellulose, hydroxypropylcellulose and other polysaccharides, in particular chitosans, hyaluronic acids, alginates, gelatins, chitins, heparins, dextrans; chondroitin sulphates; (poly)peptides/proteins, in particular collagens, fibrins, elastins, albumin; polyesters, in particular polylactides, polyglycolides, polycaprolactones; and polynucleotides. Preferably, the formulation comprises at least one polymer selected from polyvinylpyrrolidone, polyethylene oxide (PEO/PEG) and polypropylene oxide.

In particular, for a reduced adherence of bacteria to the coating, the formulation respectively coating preferably comprises a polyethylene oxide. Thus, such polymer may contribute to a further enhanced antimicrobial effect, in combination with the antimicrobial activity resulting from the release of silver ions.

In particular for polyvinylpyrrolidone (PVP) and polymers of the same class, a polymer having a molar mass corresponding to at least K15, more in particular K30, even more in particular K80 is preferred. Particular good results have been achieved with a polymer having a molar mass corresponding to at least K90. Regarding the upper limit, a K120 or less, in particular a K100 is preferred. The K-value is the value as determinable by the Method W1307, Revision 5/2001 of the Viscotek Y501 automated relative viscometer. This manual may be found at www.ispcorp.com/products/hairscin/index_{—}3.html.

The concentration of the hydrophilic polymer in the (dry) coating is usually at least 1 wt. %, in particular at least 2 wt. %, preferably at least 10 wt. %, based upon the total weight of the dry coating. Usually the concentration is up to 90 wt. % although its concentration may be higher. Preferably, the concentration is up to 80 wt. %, in particular up to 70 wt. %, up to 60 wt. % or up to 50 wt. %.

In the coating, the presence of a polyelectrolyte (which may be a further hydrophilic polymer) is preferred for its beneficial effect on the dry-out time. The use of a compound capable of forming a radical upon radiation has in particular been found advantageous in improving the lubriciousness/dry-out time of a coating comprising a polyelectrolyte, in particular a coating comprising both a polyelectrolyte and a hydrophilic polymer mentioned above.

Herein a polyelectrolyte is defined as a polymer, which may be linear, branched or cross-linked, composed of macromolecules comprising constitutional units, in which between 5 and 100% of the constitutional units contain ionic or ionisable groups, or both. A constitutional unit may be a repeating unit, e.g. a monomer.

The polyelectrolyte preferably has a number average molar mass in the range of 1 000 to 5 000 000 g/mol, as determined by light scattering.

Examples of ionic or ionisable groups that may be present include amine groups, ammonium groups, phosphonium groups, sulphonium groups, carboxylic acid groups, carboxylate groups, sulphonic acid groups, sulphate groups, sulphinic acid groups, phosphonic acid groups, phosphinic acid groups and phosphate groups.

Preferably a polyelectrolyte is selected from the group consisting of (salts of) homopolymers and copolymers of acrylic acid, methacrylic acid, acrylamide, maleic acid, sulfonic acid, styrenic acid, fumaric acid, quaternary ammonium salts and mixtures and/or derivatives thereof.

If present, the concentration of the polyelectrolyte is usually in the range of 1 to 90 wt. %. Preferably it is at least 5 wt. %, in particular at least 10 wt. %. Preferably the concentration is up to 50 wt. %, more preferably up to 30 wt. %. The weight percentages are based upon the dry weight of the coating.

The polyelectrolyte is preferably present in combination with a hydrophilic polymer that is essentially free of ionic groups (such as PVP or another non-ionic/ionisable hydrophilic polymer mentioned above. Herein the other polymer may serve as a hydrophilic supporting network for the polyelectrolyte. An advantage thereof is an increased stability of the coating. In particular the tendency of the polyelectrolyte to leak out of the coating is thus reduced. Further, a combination of two or more of such polymers is advantageous with respect to both lubricity (in particular smoothness) and dry-out time.

The weight to weight ratio of polyelectrolyte to other hydrophilic polymer is preferably in the range of 1:90 to 9:1, more preferably 1:30 to 1:1, even more preferably 1:10 to 1:5.

Optionally, the formulation comprises a cross-linker. The cross-linker may affect one or more properties of a coating prepared from the formulation. In particular, it may contribute to the formation of a polymer network which allows modulating the release pattern of silver and/or another antimicrobial agent. Further, the cross-linker may help to form a coating with a reduced tendency to leach one or more components that should remain in the coating (such as a polyelectrolyte), out of the coating. Further, the attachment of the coating to the article may be improved.

A cross-linker usually is a compound which comprises two or more functional groups—such as radically polymerizable groups. Such radically reactive polymerizable groups may be selected from the group consisting of alkenes, amino, amido, sulfhydryl (SH), unsaturated esters, unsaturated urethanes, unsaturated ethers, unsaturated amides, and alkyd/dry resins.

Particularly suitable are cross-linkers comprising vinyl groups. Such a cross-linker may be represented by the general formula G-(CR═CH₂)ₙ, wherein G can in principle by any moiety—in particular any optionally substituted hydrocarbon which may comprise one or more hetero atoms—to which vinyl groups can be bound, n is the number of vinyl groups, and R is hydrogen or a group selected from substituted and unsubstituted hydrocarbons which optionally contain one or more heteroatoms, in particular hydrogen or CH₃.

In one embodiment of the invention G is a residue of a polyfunctional compound having at least n functional groups, preferably chosen from the group consisting of polyethers, poly(meth)acrylates, polyurethanes, polyepoxides, polyamides, polyacrylamides, polyacrylics, poly(meth)acrylonics, polyoxazolines, polyvinylalcohols, polyethyleneimines and polysaccharides (such as cellulose, starch and the like) including copolymers thereof. G is more preferably an oligomer or a polymer comprising at least one polyethylene oxide and/or at least one polypropylene oxide. Such a polymer may contribute to reduced fouling of the coating, which may be beneficial with respect to an antimicrobial property of the coating. Particularly suitable are cross-linkers comprising at least one urethane group and at least one (meth)acrylate group, preferably a methacrylate group, i.e. urethane (meth)acrylates, preferably urethane methacrylates, because of their relatively high hydrolytic stability.

Because of the hydrolytic stability, the use of urethane (meth)acrylates, in particular urethane methacrylates, also offers advantages in other hydrophilic coatings, i.e. not comprising Ag particles. The invention therefore also relates to a formulation comprising a hydrophilic polymer, preferably chosen from the group of hydrophilic polymers defined above; a photo-initiator; a urethane (meth)acrylate, preferably a urethane methacrylate, and a carrier liquid. The urethane (meth)acrylate may be any molecule comprising at least one urethane group and at least one (meth)acrylate group. Suitable urethane (meth)acrylates can for example be prepared by reacting a polyol, for example a polyether polyol, with a compound comprising at least one (meth)acrylate group and at least one isocyanate group, or with a polyisocyanate and a compound containing at least one (meth)acrylate group and at least one hydroxyl group, as illustrated in the examples.

The cross-linker concentration may be chosen within wide limits, depending upon the intended result. In particular, it may be present in a concentration to provide a weight to weight ratio of the hydrophilic polymer to cross-linker in the range of 1:9 to 9:1.

The particles comprising metallic silver may be selected from particles essentially consisting of metallic silver, silver alloy particles, and metallic silver on a particular carrier, such as a ceramic material. In particular, good results have been achieved with particles essentially consisting of metallic silver.

The dimensions of the particles may be chosen within wide limits, inter alia depending upon the intended thickness of the coating, desired lubricity and/or desired wear resistance.

In general, the particle size should be less than the intended thickness of the coating. For a good lubricity and/or wear resistance, the particle size preferably is less than half the intended thickness of the coating. In absolute terms, a particle size of 3 μm or less, in particular of 2 μm or less, more in particular of 1 μm, even more in particular of 500 nm or less is preferred for good lubricity and/or wear resistance. The particle size may be determined by dynamic light scattering (in the formulation) and/or scanning electron microscopy (in the coating or the formulation).

It is further contemplated that a relatively large particle diameter is beneficial with respect to the ease of curing, especially if the intended coating is relatively thick. Without being bound by theory, it is considered that, at a given amount of particles, electromagnetic radiation (used for curing) shows less interference with the particles, if the particles are relatively large.

Relatively large particles may further be advantageous in that such particles are suitable as X-ray contrasting compound.

It is further contemplated that relatively large particles may provide a prolonged and/or constant release compared to relatively small particles.

In view of one or more of these considerations, the lower limit for the particles size may be at least 1 nm, at least 10 nm, at least 25 nm, at least 50 nm or at least 100 nm.

The concentration of particles comprising metallic silver in the formulation respectively coating may be chosen within wide limits. A metallic silver concentration of about 0.5 wt. %, based on dry weight, or more is sufficient to provide a substantial silver release, and, if desired, even a substantially constant silver release for a period of about 30 days or more. In particular, the silver concentration may be at least 1 wt. %, more in particular at least 2 wt. %, even more in particular at least 4 wt. %, based on dry weight. A relatively high silver concentration is in particular preferred for prolonging the duration of the release.

For practical reasons, in particular for allowing efficient curing of the formulation under the influence of light, the concentration of the particles comprising metallic silver is preferably 20 wt. % or less, in particular about 15 wt. % or less.

As a photo-initiator, in principle any photo-initiator can be used that is suitable to cure the formulation in the presence of electromagnetic radiation, in particular UV, visible or IR light.

Particularly suitable is a photo-initiator that is soluble in the carrier liquid, at the concentration wherein the initiator is present in the formulation.

Particularly suitable is a photo-initiator, capable of performing a photochemical homolytic bond cleavage, such as a Norrish type I cleavage reaction, or a heterolytic bond cleavage, in particular a Norrish type II cleavage.

Norrish Type I photo-initiators cause homolytic cleavage of the chromophore directly to generate radicals that initiate polymerization. Norrish Type II photoinitiators generate radicals indirectly by hydrogen abstraction from a suitable synergist, e.g. a tertiary amine. More in detail: free-radical photoinitiators are generally divided into two classes according to the process by which the initiating radicals are formed. Compounds that undergo unimolecular bond cleavage upon irradiation are termed Norrish Type I or homolytic photoinitiators, as shown by formula (1):

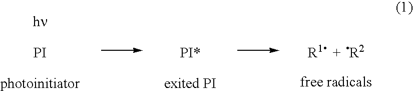

Depending on the nature of the functional group and its location in the molecule relative to the carbonyl group, the fragmentation can take place at a bond adjacent to the carbonyl group (α-cleavage), at a bond in the (β-position (β-cleavage) or, in the case of particularly weak bonds (like C—S bonds or O—O bonds), elsewhere at a remote position. The most important fragmentation in photoinitiator molecules is the α-cleavage of the carbon-carbon bond between the carbonyl group and the alkyl residue in alkyl aryl ketones, which is known as the Norrish Type I reaction.

If the excited state photoinitiator interacts with a second molecule (a coinitiator COI) to generate radicals in a bimolecular reaction as shown by formula (2), the initiating system is termed a Type II photoinitiator. In general, the two main reaction pathways for Type II photoinitiators are hydrogen abstraction by the excited initiator or photoinduced electron transfer, followed by fragmentation. Bimolecular hydrogen abstraction is a typical reaction of diaryl ketones. Photoinduced electron transfer is a more general process, which is not limited to a certain class of compounds.

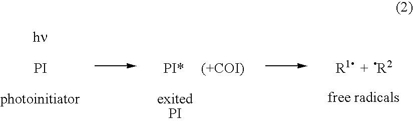

Examples of suitable Type I or cleavage free-radical photoinitiators are benzoin derivatives, methylolbenzoin and 4-benzoyl-1,3-dioxolane derivatives, benzylketals, α,α-dialkoxyacetophenones, α-hydroxy alkylphenones, α-aminoalkylphenones, acylphosphine oxides, bisacylphosphine oxides, acylphosphine sulphides, halogenated acetophenone derivatives, and the like. Commercial examples of suitable Type I photoinitiators are Irgacure 2959 (2-hydroxy-4′-(2-hydroxyethoxy)-2-methyl propiophenone), Irgacure 651 (benzildimethyl ketal or 2,2-dimethoxy-1,2-diphenylethanone, Ciba-Geigy), Irgacure 184 (1-hydroxy-cyclohexyl-phenyl ketone as the active component, Ciba-Geigy), Darocur 1173 (2-hydroxy-2-methyl-1-phenylpropan-1-one as the active component, Ciba-Geigy), Irgacure 907 (2-methyl-1-[4-(methylthio)phenyl]-2-morpholino propan-1-one, Ciba-Geigy), Irgacure 369 (2-benzyl-2-dimethylamino-1-(4-morpholinophenyl)-butan-1-one as the active component, Ciba-Geigy), Esacure KIP 150 (poly {2-hydroxy-2-methyl-1-[4-(1-methylvinyl)phenyl]propan-1-one}, Fratelli Lamberti), Esacure KIP 100 F (blend of poly {2-hydroxy-2-methyl-1-[4-(1-methylvinyl)phenyl]propan-1-one} and 2-hydroxy-2-methyl-1-phenyl-propan-1-one, Fratelli Lamberti), Esacure KTO 46 (blend of poly {2-hydroxy-2-methyl-1-[4-(1-methylvinyl)phenyl]propan-1-one}, 2,4,6-trimethylbenzoyldiphenyl-phosphine oxide and methylbenzophenone derivatives, Fratelli Lamberti), acylphosphine oxides such as Lucirin TPO (2,4,6-trimethylbenzoyl diphenyl phosphine oxide, BASF), Irgacure 819 (bis(2,4,6-trimethylbenzoyl)-phenyl-phosphine-oxide, Ciba-Geigy), Irgacure 1700 (25:75% blend of bis(2,6-dimethoxybenzoyl)2,4,4-trimethyl-pentyl phosphine oxide and 2-hydroxy-2-methyl-1-phenyl-propan-1-one, Ciba-Geigy), and the like. Also mixtures of type I photoinitiators can be used. For colored (e.g. pigmented) systems, phosphine oxide type photoinitiators and Irgacure 907 are preferred.

Preferred photoinitiators are soluble in the carrier liquid or can be adjusted to become soluble in the carrier liquid. Also preferred photoinitiators are polymeric or polymerisable photoinitiators.

Good results have been achieved with a Norrish type II initiator. Particular good results have been achieved with benzophenone. Other examples of suitable initiators include hydroxymethylphenylpropanone, dimethoxyphenylacetophenone, 2-methyl-I-4-(methylthio)-phenyl-2-morpholino-propanone-1,1-(4-isopropylphenyl)-2-hydroxy-2-methylpropan-1-one, 1-(4-dodecyl-phenyl)-2-hydroxy-2-methylpropan-1-one, diethoxyphenyl acetophenone, and the like. Phosphine oxide photoinitator types (e.g., Lucirin TPO by BASF) such as benzoyl diaryl phosphine oxide photoinitiators may be used.

The concentration of the photo-initiator can be determined based upon the efficiency of the initiator, the desired degree of polymerization and the amount of polymer (i.e. the hydrophilic polymer, if present the cross-linker and if present the polymeric polyelectrolyte).

Usually, the total initiator concentration is up to 10 wt. %, based on the total weight of the polymer. In particular in case a high dry-out time and/or high lubricity are desired, preferably a relatively low amount of photo-initiator is used, in particular an amount of up to 5 wt. %, more in particular of up to 4 wt. %. Particularly good results have been achieved with an amount of about 2 wt. % or less, for instance about 1 wt. %.

Usually the concentration is at least 0.1 wt. %, based on the weight of the polymer. For improved adhesion to the surface of the article and/or for a low amount of extractables, a relatively high concentration may be desired, in particular of at least 0.5 wt. %, more in particular of at least 1.0 wt. %, based on the weight of the polymer.

In order to facilitate dispersing the particles comprising metallic silver, to improve storage stability and/or to modulate the release of silver ions form the particles, the formulation may comprise one or more dispersing aids, in particular one or more complexing agents capable of forming a complex with silver particles or silver ions. These complexing agents can be monomeric or polymeric.

Suitable complexing agents in particular include inorganic complexing agents such as halogen ions, NH₃ and in particular ammonium salts of halogen ions such as ammonium chloride; and anions of organic acids, such as citrate or lactate; and other complexing agents capable of forming a reversible complex with ionic silver, such as polymers like polyacrylic acid, polyacrylamide and polyvinylpyrrolidone, and in particular such agents having a complexation constant in the same order of magnitude as any of the above mentioned complexing agents. A concentration may be chosen within wide ranges, in particular within the range of 0.5 to 30 wt. %, based on dry weight. A particularly effective concentration may be determined based on the concentration and the size of the particles comprising silver.

In addition to the metallic silver, an antimicrobial metal salt may be present in a formulation respectively coating of the invention. Such metal salt may be used to modulate the release pattern of metal ions. In particular, the metal ion salt may be used to realise a high release in the initial period after starting the use of the coated article. Suitable metal salts in particular include silver salts, copper salts, gold salts and zinc salts. Preferred are bromide salts and iodide salts, as bromide and iodide also have an antimicrobial activity. A concentration may be chosen within wide ranges, in particular within the range of 0.5 to 15 wt. %, based upon dry weight, more in particular in the range of 1 to 10 wt. %, based on dry weight.

Optionally one or more additives may be present in a formulation respectively coating of the invention. Such additives may in particular be selected from antioxidants, surfactants, UV-blockers, stabilisers such as anti-sagging agents, discolourants, lubricants, plasticizers, organic antimicrobial compounds, pigments, and dyes. Such components may be selected from those known in the art, e.g. the prior art identified above. If present, the total concentration of such additives is usually less than 10 wt. % based on dry weight, in particular 5 wt. % or less.

Suitable antioxidants in particular include anti-oxidative vitamins (such as vitamin C and vitamin E) and phenolic antioxidants.

The surfactant may be an ionic (anionic/cationic), non-ionic or amphoteric surfactant. Examples of ionic surfactants include alkyl sulphates (such as sodium dodecylsulphates), sodium cholate, bis(2-ethylhexyl)sulphosuccinate sodium salt, quaternary ammonium compounds, such as cetyltrimethylammonium bromide or chloride, lauryldimethylamine oxide, N-lauroylsarcosine sodium salt and sodium deoxycholate. Examples of non-ionic surfactants include alkylpolyglucosides, branched secondary alcohol ethoxylates, octylphenol ethoxylates. If present, the surfactant concentration is usually 0.001-1 wt. %, preferably 0.05-0.5 wt. % of the liquid phase.

The formulation further comprises a carrier liquid in a sufficient amount to disperse or dissolve the other components of the formulation. The carrier liquid concentration is usually at least 68 wt. %, preferably at least 75 wt. %, more preferably at least 80 wt. %, even more preferably at least 85 wt. % of the total weight of the composition. In view of handling properties (low viscosity) and/or in order to facilitate the application of the composition such that a coating with the desired thickness is obtained, the amount of solvent in the composition is preferably relatively high. For that reason the total solids content is preferably 20 wt. % or less.

The carrier liquid may be a single solvent or a mixture. It is chosen such that the polymers can be dissolved or at least dispersed therein. In particular for dissolving or dispersing the hydrophilic polymer well, it is preferred that the carrier liquid is a polar liquid. In particular, a liquid is considered polar if it is soluble in water. Preferably it comprises water and/or an organic liquid soluble in water, preferably an alcohol, more preferably a C1-C4 alcohol, in particular methanol and/or ethanol. In case of a mixture, the ratio water to organic solvent, in particular one or more alcohols, may be in the range of about 25:75 to 75:25, in particular 40:60 to 60:40, more in particular 45:55 to 55:45.

As described above, the invention further relates to a method for coating an article and to a coated article. In principle, the formulation can be used to provide any article with an antimicrobial coating. In particular, the formulation may be used to coat an article and the article is a medical device. More in particular, the article may be intended for use in an orifice of a subject, such as the ear, the mouth, the nose or the urethral tract.

Preferred coated articles of the invention include catheters, endoscopes, laryngoscopes, tubes for feeding or drainage or endotracheal use or oesophageal use, guide wires, condoms, gloves, wound dressings, contact lenses, implants, extracorporeal blood conduits, bone screws, membranes (e.g. for dialysis, blood filters, devices for circulatory assistance), sutures, fibers, filaments and meshes.

As the invention provides a coating from which silver ions can be released for a relatively long time, the invention may advantageously be used in an indwelling application, i.e. wherein the article, such as a catheter, is in contact with a tissue and/or a body fluid of a subject for a relatively long time, for example for more than a few hours to days, weeks or months (temporary) or years (permanent). The article may even be used for about a month or longer, whilst continuing to release ionic silver, before being removed.

The antimicrobial coating may be present on an inner surface (in case of a hollow article, such as a tube) and/or an outer surface. In view of providing an antimicrobial function, it is preferred that at least the surface or surfaces which are intended to be in direct contact with a body tissue and/or a body fluid are provided with the antimicrobial lubricious coating comprising metallic silver particles.

The surface to be coated can in principle be composed of any material, in particular of any polymer having satisfactory properties for the purpose of the article. Suitable polymers in particular include PVC, polytetrafluorethylene (PTFE, e.g. Teflon®), latex, silicone polymers, polyesters, polyurethanes, polyamides, polycarbonates, polyolefines, in particular ultra high molecular weight polyethylene, and the like.

If desired, the surface can be pre-treated in order to improve adherence of the antimicrobial coating, for instance a chemical and/or physical pre-treatment. Suitable pre-treatments are known in the art for specific combinations of materials for the surface of the article and the hydrophilic polymer. Examples of pre-treatments include plasma treatment, corona treatment, gamma irradiation, light irradiation, chemical washing, polarising and oxidating.

In an embodiment, the surface of the article is first provided with a primer layer, upon which the antimicrobial coating is applied to provide a coated article according to the invention. For instance, a primer layer as described in WO 06/056482, of which the contents with respect to the primer layer are incorporated herein by reference.

Application of the formulation of the invention may be done in a manner per se. Curing conditions can be determined, based on known curing conditions for the photo-initiator and polymer or routinely be determined.

In general, curing may be carried out at ambient temperature (about 25° C.) or below, although in principle it is possible to cure at an elevated temperature (for instance up to 100° C., up to 200° C. or up to 300° C.) as long as the mechanical properties or another property of the article and the coating are not adversely affected to an unacceptable extent. A reason for curing at an elevated temperature may be an improved adherence of the coating to the surface of the article.

Intensity and wavelength of the electromagnetic radiation can routinely be chosen based on the photo-initiator of choice. In particular, a suitable wavelength in the UV, visible or IR part of the spectrum may be used.

The invention will now be illustrated by the following examples.

### EXAMPLE 1

### Formulation Examples

In particular a formulation of the invention may comprise the following components within the specified usual range, respectively preferred range. For the individual components usual and preferred lower respectively upper limits may be combined with each other and/or with one or more usual and preferred lower respectively upper limits mentioned in the description above and/or in the claims.

TABLE 1

| | usual range | preferred range |
|---|---|---|
| | (wt. % based on | (wt. % based on |
| Component | dry weight) | dry weight) |
| hydrophilic polymer, | 30-99 | 40-90 |
| polyelectrolyte (optional) | | |
| and cross-linker (optional), | | |
| taken together | | |
| photo-initiator | 0.5-10 | 1-5 |
| silver particles | 0.5-20 | 4-15 |
| antimicrobial metal salt | 0-20 | 0.5-10 |
| dispersing aid | 0-30 | 1-20 |
| further additives | 0-10 | 1-5 |

The carrier liquid is present in a suitable amount to dissolve or disperse the other ingredients. Usually the concentration is at least 68 wt. %, in particular at least 80 wt. %, more in particular at least 85 wt. %.

### EXAMPLE 2

### Synthesis of Cross-Linkers

### a) Synthesis of PTGL₁₀₀₀(TDI-HEA)₂.

In a dry inert atmosphere toluene diisocyanate (TDI, Aldrich, 95% purity, 87.1 g, 0.5 mol), Irganox 1035 (Ciba Specialty Chemicals, 0.58 g, 1% (w/w) relative to hydroxy ethyl acrylate (HEA)) and tin(II) 2-ethyl hexanoate (Sigma, 95% purity, 0.2 g, 0.5 mol) were placed in a 1 liter flask and stirred for 30 minutes. The reaction mixture was cooled to 0° C. using an ice bath. HEA (Aldrich, 96% purity, 58.1 g, 0.5 mol) was added drop-wise in 30 min, after which the ice bath was removed and the mixture was allowed to warm up to room temperature. After 3 h the reaction was complete. Poly(2-methyl-1,4-butanediol)-alt-poly(tetramethyleneglycol) (Hodogaya, Mn 1000 g/mol, PTGL, 250 g, 0.25 mol) was added dropwise in 30 min. Subsequently the reaction mixture was heated to 60° C. and stirred for 18 h, upon which the reaction was complete as indicated by GPC (showing complete consumption of HEA), IR (displayed no NCO related bands) and NCO titration (NCO content below 0.02%, w/w).

b) Synthesis of PEG-di(urethane methacrylate); PEG(UMA)₂.

50.2 g (24.6 mmol OH) of PEG (Mₙ, 2040 g/mol; Biochemika Ultra from Fluka) was azeotropically distilled under nitrogen in 200 mL toluene containing 0.1 g Irganox 1035. After stirring for a night, 0.0975 g stannous octoate (Mᵣ 405.11; Aldrich) was added under nitrogen at 43° C. A solution of 8.40 g karenz MOI (Mᵣ 155.17; Showa Denko) in 20 mL of dry toluene was added dropwise to the reaction mixture in 40 minutes under stirring. After stirring the reaction mixture for an additional 3.5 hours at 43° C., an aliquot was taken to check the conversion by NMR (with addition of TFAA). In the case of a good conversion, the reaction mixture was concentrated under vacuum to a volume of approximately 120 mL. The product was collected by precipitation in diethyl ether followed by filtration. The product was additionally washed with diethyl ether and dried at room temperature under vacuum (400 mbar).

### EXAMPLE 3

### Composition of Primer Formulation

The composition of the primer formulation is given in Table 2.

TABLE 2
Composition of the primer formulation.

| | concentration |
|---|---|
| | (wt. % based on |
| Compound | total weight) |
| PTGL(TDI-HEA)₂ | 5.03 |
| PVP | 0.89 |
| Irgacure 2959 | 0.24 |
| Ethanol | 93.84 |

### EXAMPLE 4-10

### Composition of Top Coat Formulation

TABLE 3
Composition of top coat formulation of Examples 4-10.

| | Ex 4 | Ex 5 | Ex 6 | Ex 7 | Ex 8 | Ex 9 | Ex 10 | Ex 19 |
|---|---|---|---|---|---|---|---|---|
| Compound | Amount (%, w/w) | | | | | | | |
| PVP 1.3M (Povidone, | 6.13 | 6.10 | 6.10 | 6.13 | 3.69 | 4.91 | 6.14 | 3.14 |
| Sigma-Aldrich) | | | | | | | | |
| Benzophenone(Sigma- | 0.06 | 0.06 | 0.06 | 0.06 | 0.06 | 0.06 | 0.06 | — |
| Aldrich) | | | | | | | | |
| Irgacure 2959 (Sigma- | — | — | — | — | 0.06 | — | — | 0.09 |
| Aldrich) | | | | | | | | |
| Nanosilver (QSI) | 0.55 | 0.55 | 0.55 | 0.55 | 0.55 | 0.55 | 0.55 | — |
| Distilled water | 46.63 | 46.34 | 46.34 | 46.60 | 46.58 | 46.63 | 46.62 | 45.24 |
| Methanol (Merck) | 46.63 | 46.34 | 46.34 | 46.60 | 46.58 | 46.63 | 46.62 | 45.24 |
| PEG(UMA)₂ | — | — | — | — | 2.48 | — | — | 4.71 |
| Tween 80 (Sigma- | — | — | — | 0.06 | — | — | — | — |
| Aldrich) | | | | | | | | |
| Poly(acrylamide-co- | — | — | — | — | — | 1.22 | — | 1.58 |
| acrylic acid). Na/20% | | | | | | | | |
| acrylamide (Sigma- | | | | | | | | |
| Aldrich) | | | | | | | | |
| 3,5-Di-tert-butyl-4- | — | — | — | — | — | — | 0.01 | — |
| hydroxybenzylalkohol | | | | | | | | |
| (Sigma-Aldrich) | | | | | | | | |
| Silver acetate (Strem | — | — | 0.61 | — | — | — | — | — |
| chemicals) | | | | | | | | |
| Ammonium chloride | — | 0.61 | — | — | — | — | — | — |
| (Merck) | | | | | | | | |
| Total | 100.00 | 100.00 | 100.00 | 100.00 | 100.00 | 100.00 | 100.00 | 100.00 |

The compounds were dissolved in the solvent under stirring at room temperature. First the compounds other than the silver particles were added to the solvent. The silver nanoparticles were only added after dissolution of the other compounds, to avoid undesirable sedimentation of particles.

### EXAMPLE 11

### PVC Catheters

Uncoated PVC tubings were used as a substrate to be coated with a lubricious anti-microbial coating. The PVC tubing had a length of 23 cm, an outside diameter of 4.65 mm (14 Fr), and an inside diameter of 3.35 mm. The tubings were closed on one side.

### EXAMPLE 12

### Coating and Curing Process

A guidewire was inserted in the tubing to fix the tubing and to attach it in the holder of the Harland PCX coater/175/24. The tubing was cleaned with lens tissues (Whatman) immersed in a 96% (w/v) aqueous ethanol solution (Merck). The assembly was subsequently dipped in the primer and the topcoat formulations using the Harland coater.

The Harland PCX coater/175/24 was equipped with a Harland Medical systems UVM 400 lamp. The intensity of the lamps of the Harland PCX coater/175/24 was on average 60 mW/cm2 and was measured using a Solatell Sola Sensor 1 equipped with an International Light detector SED005#989, Input Optic: W#11521, filter: wbs320#27794. The IL1400A instruction manual of International Light was applied, which is available on the internet: www.intl-light.com.

The tubing was dipped in the primer formulation for 10 seconds, moved up with a speed of 0.3 cm/s and cured for 15 seconds with a total dose of 0.9 J/cm². The tubing was then dipped in the topcoat formulation for 10 seconds, moved up with a speed of 1.5 cm/s and cured for 360 seconds with a total dose of 21.6 J/cm². After drying for a night at room temperature, the coatings were analysed.

The applied coating parameters are given in Table 4.

TABLE 4
Harland Coating parameters selection table

| | Primer | Topcoat | Range | |
|---|---|---|---|---|
| Dipping Cycle | | | | |
| Move device carrier to | 117 | 117 | 2 to 175 | cm |
| position | | | | |
| speed (cm/sec) | 6.5 | 6.5 | 0.2 to 6.5 | cm/sec |
| acceleration (sec) | 0.1 | 0.1 | 0.1 | cm/sec/sec |
| Operator Prompt | | | | |
| Operator Prompt | | | | |
| Move device carrier down | 7 | 7 | 2 to 175 | cm |
| speed (cm/sec) | 4 | 2 | 0.2 to 6.5 | cm/sec |
| acceleration (sec) | 0.1 | 0.1 | 0.1 | cm/sec/sec |
| Operator Prompt | | | | |
| Move device carrier down | 25 | 24.5 | 2 to 175 | cm |
| speed (cm/sec) | 2 | 2 | 0.2 to 6.5 | cm/sec |
| acceleration (sec) | 0.1 | 0.1 | 0.1 | cm/sec/sec |
| Time Pause | 10 | 10 | 0 to 1800 | sec |
| Move device carrier up | 26 | 26 | | |
| speed (cm/sec) | 0.3 | 1.5 | 0.2 to 6.5 | cm/sec |
| acceleration (sec) | 0.1 | 0.1 | 0.1 | cm/sec/sec |
| Move device carrier to | 148 | 148 | 2 to 175 | cm |
| position | | | | |
| speed (cm/sec) | 6.5 | 6.5 | 0.2 to 6.5 | cm/sec |
| acceleration (sec) | 0.1 | 0.1 | 0.1 | cm/sec/sec |
| Operator Prompt | | | | |
| Cure Cycle | | | | |
| Rotator On | 4 | 4 | 1 to 8 | rpm |
| UV lights Full Power | | | | |
| Time pause | 15 | 360 | 0 to 1800 | sec |
| Close Shutter | | | | |
| UV lights Standby Power | | | | |
| Rotator Off | | | | |

### EXAMPLE 13

### a) Lubricity and Wear Test

Lubricity and wear tests were performed on a Harland FTS5000 Friction Tester (HFT). The protocol was selected: see Table B for HFT settings. Friction tester pads were used from Harland Medical Systems, P/N 102692, FTS5000 Friction Tester Pads, 0.125*0.5**0.125, 60 durometer. Subsequently the desired test description was inserted when “run test” was activated. After inserting the guidewire into the catheter, the catheter was attached in the holder. The device was adjusted down to the desired position such that the catheter was soaked in demineralised water for 1 min. After zero gauging in water the protocol was activated by pushing “start”. The data were saved after finishing. The holder was removed from the force gauge and subsequently the catheter was removed from the holder.

TABLE 5
HFT settings

| | |
|---|---|
| Transport movement (cm) | 10 |
| Clamp force (g) | 300 |
| Pull speed (cm/s) | 1 |
| Acceleration time (s) | 2 |
| Number of rubs | 25 |

### b) Determination of Dry-Out Time.

Dry-out time is herein defined as the maintenance of lubricity of the lubricious coating on the coated PVC catheter as a function of time, which is determined by measuring the friction in g as a function of time on a Harland FTS Friction Tester (HFT). After inserting the guidewire into the coated PVC catheter, the catheter was attached in the holder. The catheter was soaked in demineralised water for 1 min. The holder with the catheter was put in the force gauge and the device was jogged down to the desired position and the test was started immediately according to the same settings as for the lubricity test. Measurements were performed after 1, 2, 5, 7.5, 10, 12.5 and 15 minutes. The friction tester pads were cleaned and dried after each measurement. The data were saved after finishing. The holder was removed from the force gauge and subsequently the catheter was removed from the holder.

### EXAMPLE 14

### Quantification of Silver Ion Release

FIG. 1 schematically shows the set up used to determine the silver ion release.

Eight pieces of coated catheter (12 cm each) were put on a 2 mm glass rod. The rod was inserted into a glass flow chamber and fixed in position with a glass stopper. The chamber was filled with a 10 mM potassium phosphate buffer, 150 mM NaCl, pH 7.0 (PBS buffer, Merck). A flow of buffer solution was subsequently applied to the flow chamber (0.7 mL/min) using a Gilson 307 HPLC pump. The eluate was collected (60 min per fraction) by means of a Lambda Omnicoll fraction collector. For analysis the fractions were acidified with HNO₃ (Merck suprapur 65%) to pH 1.

Samples were analysed using graphite furnace atomic absorption spectrophotometry according to DIN 38406 E18.

The results are shown in FIG. 4. These show the silver ion release data, measured by the method described in Example 14, Example 4 (−) vs the Tyco Kendall silver Foley catheter (♦).

For the Bardex catheter no silver ion release could be detected by the described method, which had a detection limit of 0.5 ppb.

### EXAMPLE 15

### Antimicrobial Activity Tests

### a) Determination of Bacterial Adhesion to and Bactericidal Activity at the Coating Surface.

The valves for a modified Robbins device (FIG. 6) were sonicated for 5 min in 2% (w/v) RBS (Omni Clean RBS 35, Omnilabo, Breda, The Netherlands), flushed with hot and cold water, dipped in methanol, flushed with distilled water, dipped in a 70% (v/v) aqueous ethanol solution and rinsed with a sterile 10 mM potassium phosphate buffer, 150 mM NaCl, pH 7.0 (PBS buffer). Catheter parts (2 cm), two of each catheter, were fixed in the valves.

Staphylococcus epidermidis 3399 was cultured from frozen stock on blood agar plates. Precultures were grown in 5 mL tryptone soy broth medium (Oxoid). A culture was grown from the preculture in 200 mL tryptone soy broth medium overnight at 37° C. The cells were harvested by centrifugation (6000 g, 5 min, 10° C.). They were washed twice and resuspended in the sterile PBS buffer to a concentration of 5×10⁸ cells/mL.

The catheter parts were inoculated with 20 mL of this bacterial suspension. After 2 h at 37° C. with shaking (60 rpm), the catheter parts were washed by dipping in sterile PBS buffer. They were subsequently placed in the modified Robbins device filled with tryptone soy broth medium. During the experiment the modified Robbins device was maintained at 37° C. and tryptone soy broth medium was perfused through the system with a flow rate of 0.4 mL/min.

After 48 h the catheter parts were removed from the modified Robbins device and dipped in sterile PBS buffer to remove the planktonic cells. The catheter parts were subsequently removed from the valves and the biofilms were stained with a Live/Dead viability kit (Molecular Probes). The stained biofilms were analysed by means of a confocal laser scanning microscope (Leica TCS SP2, Leica Microsystems) with a 40× water objective.

The results are shown in FIGS. 5A and 5B. FIG. 5A shows a CSLM image (in xy-plane) of a 2 days old S. epidermidis 3399 biofilm on the PVC tubing coated with a silver-free coating (z: 22 μm). The PVC surface is the more or less horizontal grey band in the middle section of the image; in the original colour image it was shown in green (as it has been stained with the green dye of the kit). The biofilm is located on top of the coating. The biofilm contains both dead bacteria (grey spots in lower half of the image; shown in red in the original colour image) and living bacteria (the white spots in the lower half of the image; the contrast has been adjusted manually for improved visualisation in the black and white copy of the colour image, in which the living bacteria were shown in green).

CSLM image (in xy-plane) of a 2 days old S. epidermidis 3399 biofilm on the PVC tubing coated with silver-containing coating according to the invention (z: 48 μm). The biofilm is located on top of the coating. A reduction of the amount of adhering bacteria can be observed, compared to the silver-free coating (FIG. 5A). Moreover, the remaining cells are dead.

### b) Determination of Bacteriocidal Activity of the Coating and Bacterial Adherence to the Coating by Plate Counting Experiments.

Bacteriocidal Activity Test:

Escherichia coli ATCC 11105 was cultured from frozen stock in sterile Luria-Bettani medium. The bacterial suspension had a concentration of about 2.3×10¹⁰ CFU/mL. It is noted that his concentration is considerably higher than a typical concentration for a beginning infection in vivo (10³-10⁴ CFU/mL).

The suspension was diluted in sterile PBS buffer to obtain a final concentration of 2.3×10⁷ CFU/mL. In 40 mL of this bacterial suspension 5 cm of a coated catheter was incubated for 24 h at 20° C. while shaking at 200 rpm. The suspension was subsequently serial diluted and plated out on petri dishes filled with Luria-Bettani agar. After incubation overnight at 37° C., the bacterial colonies formed on the agar were counted.

Control experiments (bacterial suspension in which no catheter had been incubated) and comparative experiments using respectively two uncoated PVC tubings, two coated catheters which do not contain silver and are otherwise the same as the catheters of the invention, two Bardex catheters and two Tyco Kendall catheters.

The results are shown in the following Table.

TABLE 6

| Sample | Colony forming units (CPU, log units) | | |
|---|---|---|---|
| Control I | 7.41 | 7.35 | 7.30 |
| Control II | 7.35 | 7.05 | 7.19 |
| Lubricious coating I (as | 7.34 | 7.30 | 7.38 |
| Example 4 but without | | | |
| Ag) | | | |
| Lubricious coating II | 7.34 | 7.28 | 7.33 |
| (as Example 4 but | | | |
| without Ag) | | | |
| Example 4 I | — | — | 1.0 |
| Example 4 II | — | — | 1.0 |
| Tyco Kendall I | 4.00 | 2.11 | — |
| Tyco Kendall II | — | 3.90 | 4.00 |
| Bardex I | 7.43 | 7.30 | 7.11 |
| Bardex II | 7.26 | 7.39 | 7.28 |

The three “CFU” columns show cell counts for sections in the dishes corresponding to three sections of the catheters. It is shown that only the coated article of the invention was effective in killing substantially all bacteria over the full length of the catheter. The lubricious coating without silver and the Bardex coating did not result in a substantial reduction of bacteria compared to the control. The Tyco Kendall coating seemed effective to some extent, but in both Tyco Kendall catheters a large variation was observed in the antimicrobial activity, compared to the coated articles of the invention.

Bacterial Adhesion (+Bacteriocidal Activity) Test:

Escherichia coli ATCC 11105 was cultured from frozen stock in sterile Luria-Bettani medium. The bacterial suspension had a concentration of about 2.3×10¹⁰ CFU/mL. This suspension was diluted in sterile PBS buffer to obtain a final concentration of 2.3×10⁷ CFU/mL. In 40 mL of this bacterial suspension two pieces of a coated catheter (5 cm length) were incubated for 4 h at 20° C. with shaking at 200 rpm. The catheter part was subsequently removed from the bacterial suspension and washed by dipping in sterile PBS buffer. The washed catheter parts were then rolled over Luria-Bettani agar in a petri dish and the agar with the catheter was incubated overnight at 37° C. Photographs were made to compare the amount of colonies formed on the agar for different samples.

FIG. 7A-D show respectively: A) petri dish treated with a catheter comprising a lubricious coating as described in Example 4, but without silver; B) as A, but with silver; C) petri dish treated with Bardex silver Foley catheter; D) Tyco Kendall silver Foley catheter. It is shown that the antimicrobial activity of the coating of the invention is much better than for the Tyco Kendall catheter and the Bardex catheter. In fact, the latter did not show an improvement compared to the silver-free catheter.

### EXAMPLE 16

### Lubricity and Wear Resistance; a Catheter Coated According to the Invention vs. Commercially Available Catheters

The catheter of Example 4 was compared with commercially available silver coated Foley catheters sold by Bardex and Tyco Kendall making use of the test described in Example 13a. The results are shown in FIG. 2. It is shown that not only the initial friction force of a catheter of the invention is better than for the commerically available but also that a low friction force (and thus good lubricity) is maintained for many cycles.

### EXAMPLE 17

### Lubricity and Wear Resistance: for Coated Articles of Examples 4-10

FIG. 3 shows the friction force as a function of the number of cycles in a method described in Example 13a. It is shown that good lubricity is maintained for many cycles.

### EXAMPLE 18

### Dry-Out Time

The following table shows dry-out times, measured by the method described in Example 13b, for coated PVC tubing of the invention (Examples 4-10), Tyco Kendall catheters and Bardex catheters.

TABLE 7

| Sample | dry-out time (min) |
|---|---|
| Example 4 | 10 |
| Example 5 | 20 |
| Example 6 | 15 |
| Example 7 | 20 |
| Example 8 | 5 |
| Example 9 | 25 |
| Example 10 | 20 |
| Tyco Kendall | 0 |
| Bardex | 0 |

### EXAMPLE 19

### Hydrolytic Stability of Top Coat Formulation Comprising PEG(UMA)₂

A top coat formulation comprising PEG(UMA)₂ as a cross-linker (see composition in Table 3) was placed in a brown bottle and subjected to incubation at 50° C. for 18 days. Samples were taken after 0, 2, 7 and 18 days and analyzed using HPLC-DAD-MS.

Procedure HPLC-DAD-MS: the test samples were dissolved in water (1000-2000 ppm), separated by HPLC and detected with diode array detection (DAD) and mass spectroscopy (MS). Specifications HPLC-DAD-MS:
Flow rate: 0.5 mL/min
Mobile phase: A=0.1% formic acid, B=0.1% formic acid in acetonitrile
Gradient: t=0 min: 2% B, t=5 min: 2% B, t=45 min: 98% B, t=60 min: 98% B, t=61 min: 2% B
Column temperature: 40° C.
Injection volume: 5 μL
DAD: spectra from 190 to 600 nm (2 nm step size) were stored, spectra at 195, 200, 210, 230 and 254 nm were collected
ES(+)-MS detection: m/z 50-1500, 50 V frag, 10 L/min, 50 psig neb, 350° C., 2.5 kV.

In Table 8 the amount of PEG(UMA)₂ is given as a function of incubation time and compared to the amount of polyethylene glycol diacrylate (PEG₄₀₀₀DA, from PEG (Mᵣ 3500-4500, Biochemika Ultra from Fluka) synthesis described in WO06/056482 A1).

TABLE 8
Hydrolytic stability of PEG(UMA)2 compared to PEG₄₀₀₀DA

| Incubation time | PEG(UMA)₂ (Ex 19) | PEG₄₀₀₀DA |
|---|---|---|
| 0 | 2.9 | 4.3 |
| 2 | 2.1 | 0.7 |
| 7 | 0.7 | 0 |
| 18 | 0 | 0 |

The results show that PEG(UMA)₂ has an enhanced hydrolytic stability compared to PE₄₀₀₀DA.

1. Formulation for preparing an antimicrobial hydrophilic coating, which formulation comprises a hydrophilic polymer; a photo-initiator; particles comprising metallic silver (i.e. Ag°); and a carrier liquid.

2. Formulation according to claim 1, wherein the particles have a number average diameter in the range of 1 nm to 3 μm, preferably in the range of 10 nm to 1 000 nm.

3. Formulation according to claim 1, comprising a dispersing aid for the silver particles, preferably a complexing agent capable of forming a complex with silver ions, more preferably a complexing agent selected from the group consisting of ions of a halogen, organic acids and polymeric complexing agents.

4. Formulation according to claim 1, further comprising an antimicrobial metal salt, preferably selected from silver salts, copper salts, gold salts, zinc salts.

5. Formulation according to claim 1 wherein the amount of metallic silver is 0.5 to 20 wt. %, based upon the dry weight of the formulation.

6. Formulation according to claim 1, wherein the hydrophilic polymer is cross-linkable or graftable upon photo-initiation.

7. Formulation according to claim 1, wherein the hydrophilic polymer is selected from the group consisting of poly(lactams), in particular polyvinylpyrrolidones; polyurethanes; homo- and copolymers of acrylic and methacrylic acid; polyvinyl alcohols; polyvinylethers; maleic anhydride based copolymers; polyesters; vinylamines; polyethyleneimines; polyethylene oxides; poly(carboxylic acids); polyamides; polyanhydrides; polyphosphazenes; cellulosics, in particular methyl cellulose, carboxymethyl cellulose, hydroxymethylcellulose, hydroxypropylcellulose and other polysaccharides, in particular chitosans, hyaluronic acids, alginates, gelatins, chitins, heparins, dextrans; chondroitin sulphates; (poly)peptides/proteins, in particular collagens, fibrins, elastins, albumin; polyesters, in particular polylactides, polyglycolides, polycaprolactones; and polynucleotides.

8. Formulation according to claim 1 comprising a polyelectrolyte, preferably a polyelectrolyte comprising at least one ionised or ionisable group selected from the group consisting of amine groups, ammonium groups, phosphonium groups, sulphonium groups, carboxylic acid groups, carboxylate groups, sulphonic acid groups, sulphate groups, sulphinic acid groups, phosphonic acid groups, phosphinic acid groups and phosphate groups, preferably a polyelectrolyte selected from the group consisting of homopolymers and copolymers of acrylic acid including salts thereof, methacrylic acid including salts thereof, acrylamide including salts thereof, maleic acid including salts thereof, sulfonic acid including salts thereof, quaternary ammonium salts and mixtures and/or derivatives thereof.

9. Formulation according to claim 1, comprising a cross-linker, preferably a cross-linker represented by the formula G-(CR═CH₂)ₙ, wherein G can in principle by any moiety—in particular any optionally substituted hydrocarbon which may comprise one or more hetero atoms—to which vinyl groups can be bound, n is the number of vinyl groups, and R is hydrogen or a group selected from substituted and unsubstituted hydrocarbons which optionally contain one or more heteroatoms, in particular hydrogen or CH₃.

10. Formulation according to claim 9, wherein the crosslinker is a urethane (meth)acrylate, preferably a urethane methacrylate.

11. Formulation according to claim 1 comprising at least one compound selected from antioxidants, surfactants, UV-blockers, stabilisers such as anti-sagging agents, discolourants, lubricants, plasticizers, organic antimicrobial compounds, pigments and dyes.

12. Formulation according to claim 1, wherein the liquid carrier is a polar liquid, preferably selected from the group consisting of water, water-soluble alcohols and mixtures comprising any of these.

13. Formulation comprising a hydrophilic polymer, preferably chosen from the group defined in claim 7; a photo-initiator; a urethane (meth)acrylate, preferably a urethane methacrylate, and a carrier liquid.

14. Formulation according to claim 13, wherein the urethane (meth)acrylate is prepared by reacting at least one polyol, for example a polyether polyol, with a compound comprising at least one (meth)acrylate group and at least one isocyanate group, or with a polyisocyanate and a compound containing at least one (meth)acrylate group and at least one hydroxyl group.

15. Method for preparing a formulation as defined in claim 1, comprising dissolving or dispersing
the hydrophilic polymer, -the photo-initiator,
if present, the dispersing aid
if present, the cross-linker in carrier liquid; and thereafter dispersing the silver particles.

16. Method for preparing a coated article, comprising
applying a formulation according to claim 1 to at least one surface of the article;
and allowing the formulation to cure by exposing the formulation to electromagnetic radiation thereby activating the photo-initiator.

17. An article comprising a hydrophilic coating on a surface, in particular a coated article obtainable by a method according to claim 16, wherein the coating comprises a cured hydrophilic polymer and particles comprising metallic silver (Ag°).

18. An article according to claim 17, wherein the cured polymer is a cross-linked polymer or a grafted polymer.

19. An article according to claim 16, wherein the coating is lubricious when wetted.

20. An article according to claim 17, wherein the article is a medical device, preferably selected from catheters, endoscopes, laryngoscopes, tubes for feeding or drainage or endotracheal use, guide wires, condoms, gloves, wound dressings, contact lenses, implants, extracorporeal blood conduits, bone screws, membranes (e.g. for dialysis, blood filters, devices for circulatory assistance), sutures, fibers, filaments and meshes.

21. Formulation according to claim 1 for medical use.

22. Use of a formulation according to claim 1 in the manufacture of a composition—in particular a coating—for the treatment of a disorder selected from the group consisting of complications of the urinary tract, complications of a cardiovascular vessel, kidney infections, blood infections (septicemia), urethral injury, skin breakdown, bladder stones and hematuria, or to prevent infections

23. Use of a formulation according to claim 1.
